(19) United States
(12) Patent Application Publication
(10) Pub. No.: US 2015/0289786 A1
(43) Pub. Date: Oct. 15, 2015

(54) Method of Acoustic Screening for Processing Hearing Loss Patients by Executing Computer-Executable Instructions Stored On a Non-Transitory Computer-Readable Medium

(71) Applicants: Reginald G. Garratt, Naples, FL (US); Sean R. Garratt, Naples, FL (US)

(72) Inventors: Reginald G. Garratt, Naples, FL (US); Sean R. Garratt, Naples, FL (US)

(21) Appl. No.: 14/685,586

(22) Filed: Apr. 13, 2015

Related U.S. Application Data
(60) Provisional application No. 61/978,601, filed on Apr. 11, 2014.

(51) Int. Cl. A61B 5/12; A61B 5/00

(52) U.S. Cl. CPC A61B 5/123; A61B 5/7475; A61B 5/7435; A61B 5/743; A61B 5/72; A61B 5/0004; A61B 5/486; A61B 2560/0223; A61B 2560/0475; A61B 2503/12

(57) ABSTRACT

A method of method of acoustic screening for processing hearing loss patients utilizes a computing device connected to an audio output device to administer a hearing loss exam to a patient, display an audiogram of their hearing loss to the patient, and attempt to gently guide the patient into undergoing treatment for hearing loss. A correction algorithm is produced from the audiometric data in order to present the patient with comparison scenarios from typical day-to-day sounds with and without being modified by the correction algorithm.

The current application claims a priority to the U.S. Provisional Patent application Ser. No. 61/978,601 filed on Apr. 11, 2014. The current application is filed on Apr. 13, 2015, wherein Apr. 11, 2015 and Apr. 12, 2015 were on a weekend.

### FIELD OF THE INVENTION

The present invention relates generally to hearing patient processing. More particularly, the present invention is designed to guide a hearing patient into seeking treatment for hearing loss.

### BACKGROUND OF THE INVENTION

There are many audio screening devices which enable a physician or other persons to screen a patient for hearing impairment. The proposed design performs this function, measures the hearing impairment of a patient and provides an audiogram output which can be used to evaluate the degree of hearing impairment of the patient. This is well known and established technology and the purpose of the patent application is to overcome the resistance (“denial”) of hearing impaired persons and so enhance the sale and fitting of hearing aids.

It is well known that hearing impaired patients with a significant hearing loss, generally regarded as greater than 35 dB loss in their better ear, often avoid being diagnosed because they do not wish to admit they have a hearing impairment, which may lead to them to buying and wearing hearing aids. This phase of rejection of a hearing condition is commonly referred to as; “the person being in denial”.

The device which concerns this patent application is designed to inform and guide a hearing impaired person to voluntarily ask a licensed hearing professional for an acoustic hearing evaluation.

### BRIEF DESCRIPTION OF THE DRAWINGS

FIG. 1 is a stepwise flow diagram of the general process of the present invention.

FIG. 2 is a stepwise flow diagram describing steps for executing the audiometric exam.

FIG. 3 is a stepwise flow diagram describing steps for displaying the audiogram.

FIG. 4 is a stepwise flow diagram describing steps for calibrating the computing device and the audio output device.

FIG. 5 is a stepwise flow diagram describing potential steps taken after the user receives their results from the audiometric exam.

FIG. 6 is a stepwise flow diagram describing the execution of the preferred embodiment of the present invention.

FIG. 7 is a continuation of FIG. 6.

FIG. 8 is a continuation of FIG. 7.

FIG. 9 is an example screen of an audiogram used in the present invention.

FIG. 10 is an example screen of practice instructions used in the present invention.

FIG. 11 is an example screen of a patient data screen used in the present invention.

FIG. 12 is an example screen of a test overview used in the present invention.

FIG. 13 is an example screen of test instructions used in the present invention.

FIG. 14 is an example screen of a test user interface used in the present invention.

FIG. 15 is an example screen of a test complete screen used in the present invention.

FIG. 16 is an example screen of a simplified audiogram used in the present invention.

FIG. 17 is an example screen of first conversational audio used in the present invention.

FIG. 18 is an example screen of improved conversational audio used in the present invention.

FIG. 19 is an example screen of a first news announcer audio used in the present invention.

FIG. 20 is an example screen of improved announcer audio used in the present invention.

FIG. 21 is an example screen of a confirmation question used in the present invention.

FIG. 22 is an alternative example screen of a confirmation question used in the present invention.

FIG. 23 is an example screen of NCOA information used in the present invention.

FIG. 24 is an example screen of a health warning used in the present invention.

FIG. 25 is an example screen of a book appointment screen used in the present invention.

FIG. 26 is an example screen of a final message used in the present invention.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced without the implementation of some features as they are described. It should be understood that some details have not been described in detail in order to not unnecessarily obscure focus of the invention.

The present invention is intended as a means to guide a hearing impaired person through a screening process in a private and automated manner in order to streamline patient processing and guide hearing impaired persons into voluntarily seeking treatment for hearing loss. The present invention is primarily a method, which is substantially embodied as a software application. The present invention is facilitated through a computing device, such as, but not limited to, a personal computer such as a desktop computer or a laptop computer, a mobile device such as a tablet or smartphone, or any other electronic computing device which comprises a user interface and is capable of facilitating the use of the software application for patient screening. The user interface may be, but is not limited to a touchscreen display such as that utilized by a tablet device, or a monitor and mouse/keyboard combination such as typically used with personal computers. In addition to the computing device, an audio output device must also be utilized. In the preferred embodiment of the present invention, the audio output device is a pair of headphones. The headphones should be able to accurately reproduce audio signals coming from the computing device with a desired tolerance range of +/−2 decibels in order to properly and accurately facilitate the functionality of the present invention, in addition to adequate exclusion of ambient noise. The computing device and the audio output device must be electronically connected; that is, in the preferred embodiment the headphones are plugged into a headphone connection on the computing device.

There are two phases to the audio screening test applied in the present invention; the first phase is the actual audiometric measurement which will determine if a person (patient) undergoing the test is hearing impaired and to what degree. Such audiometric data is derived from an “acoustically calibrated” system, the methods of which are described later in the application.

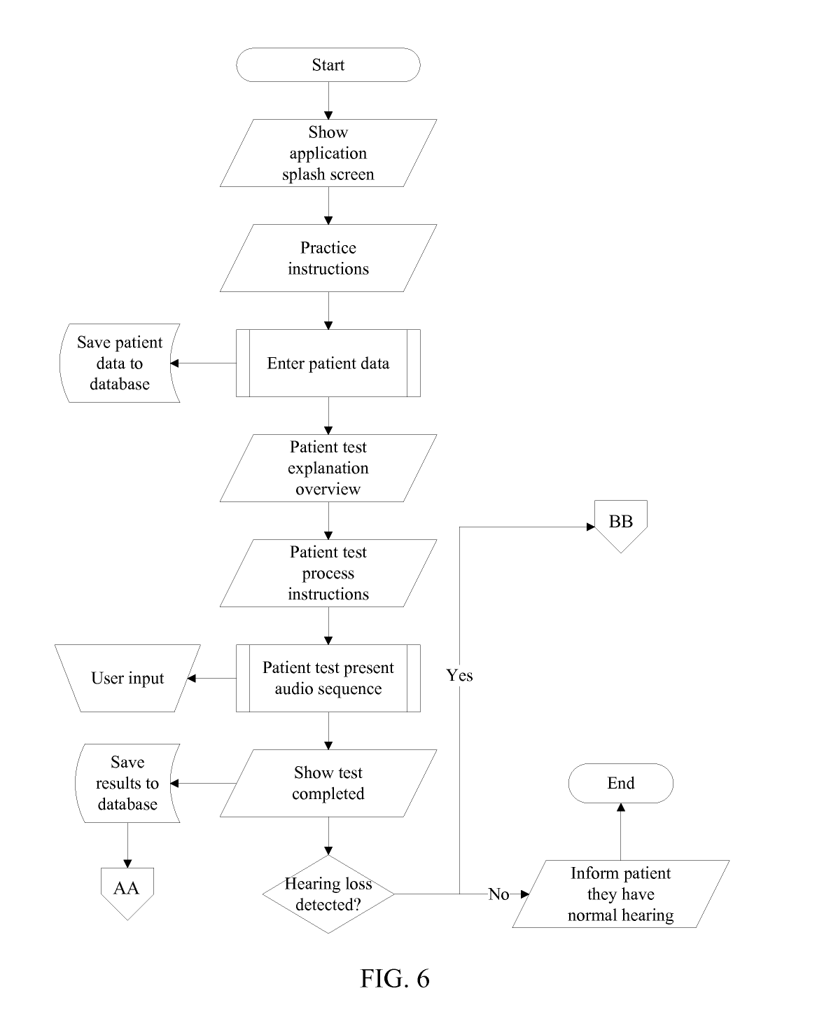

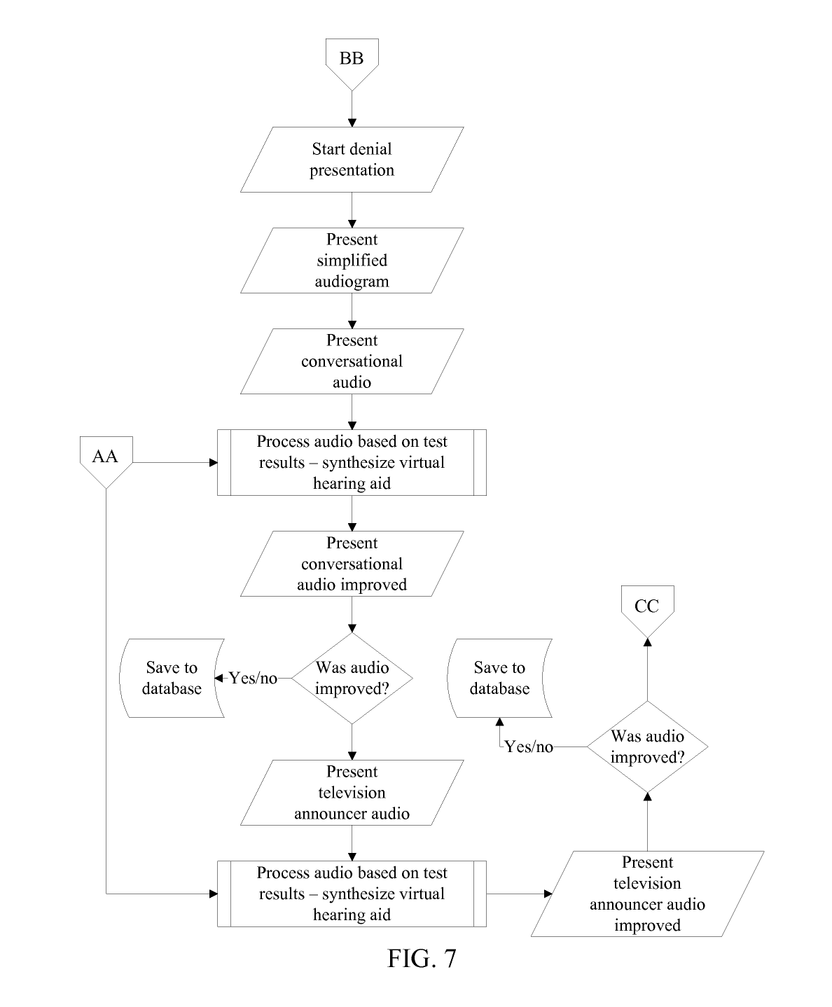

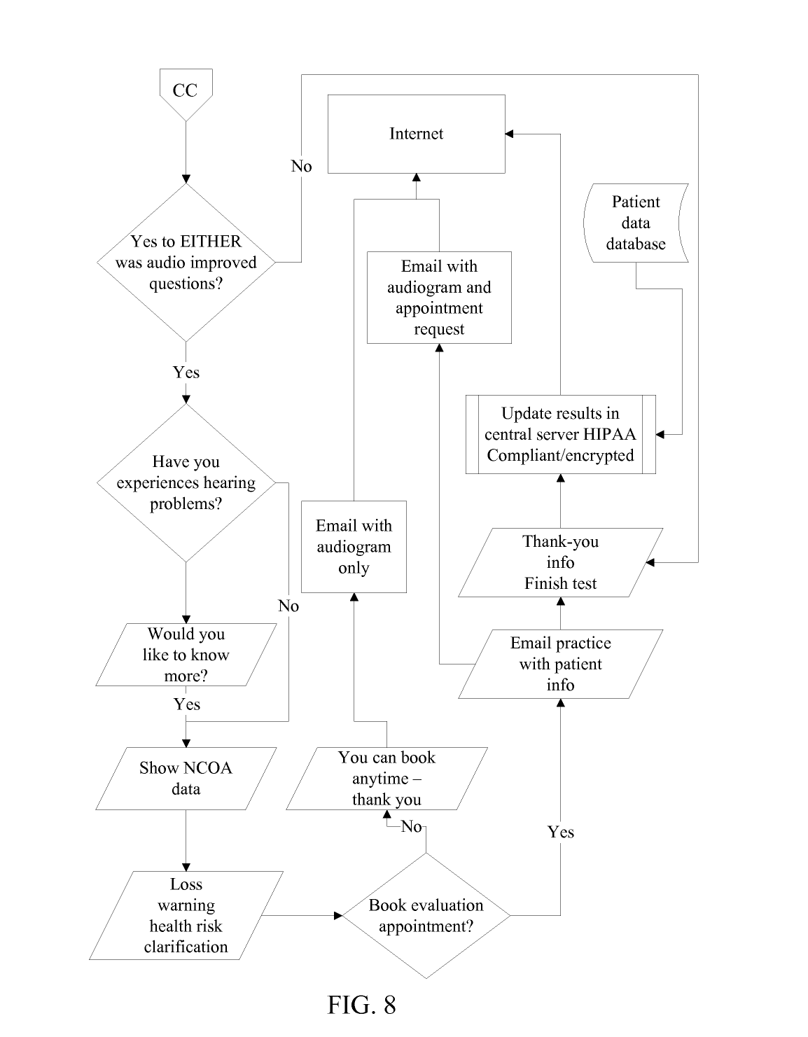

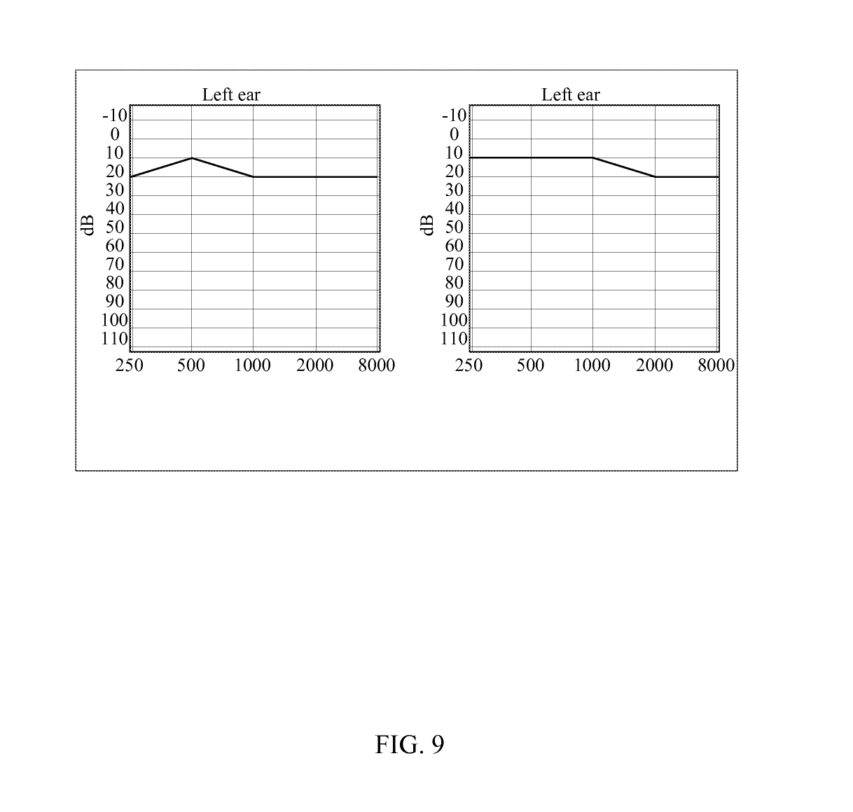

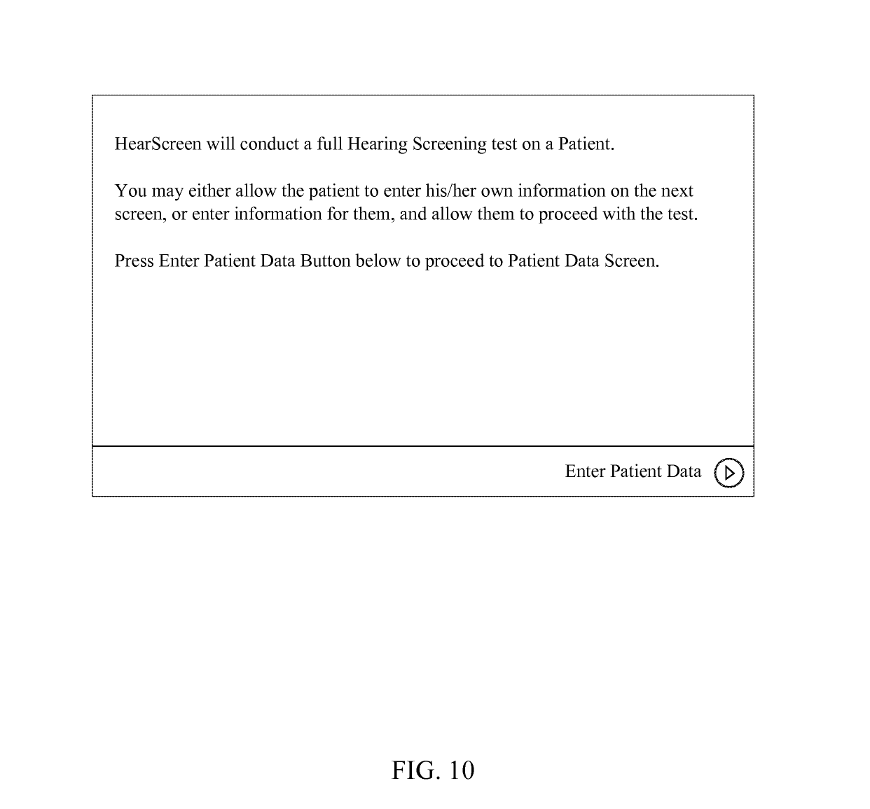

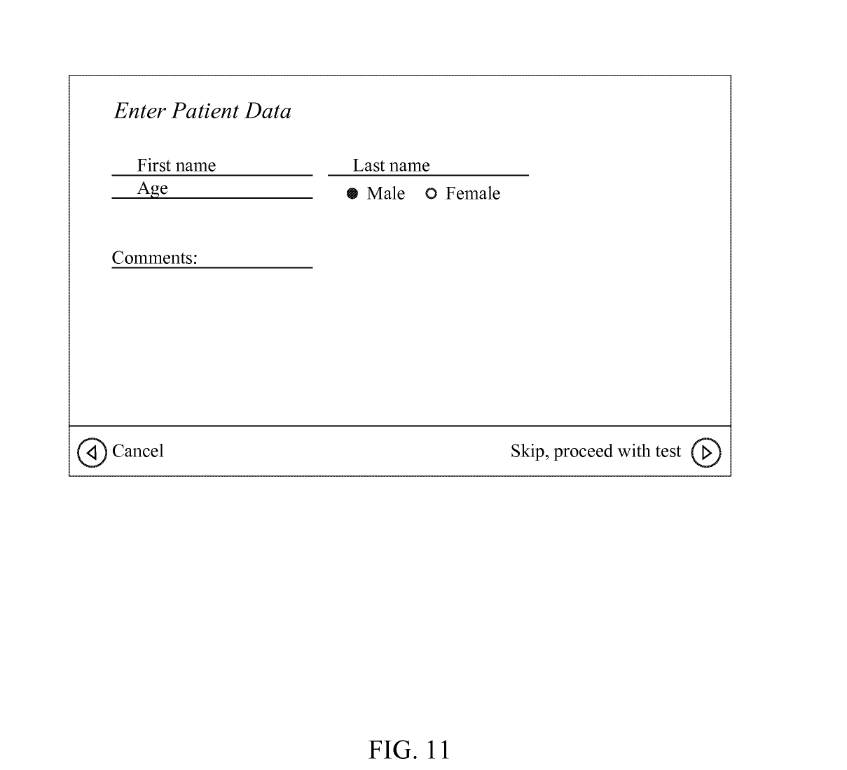

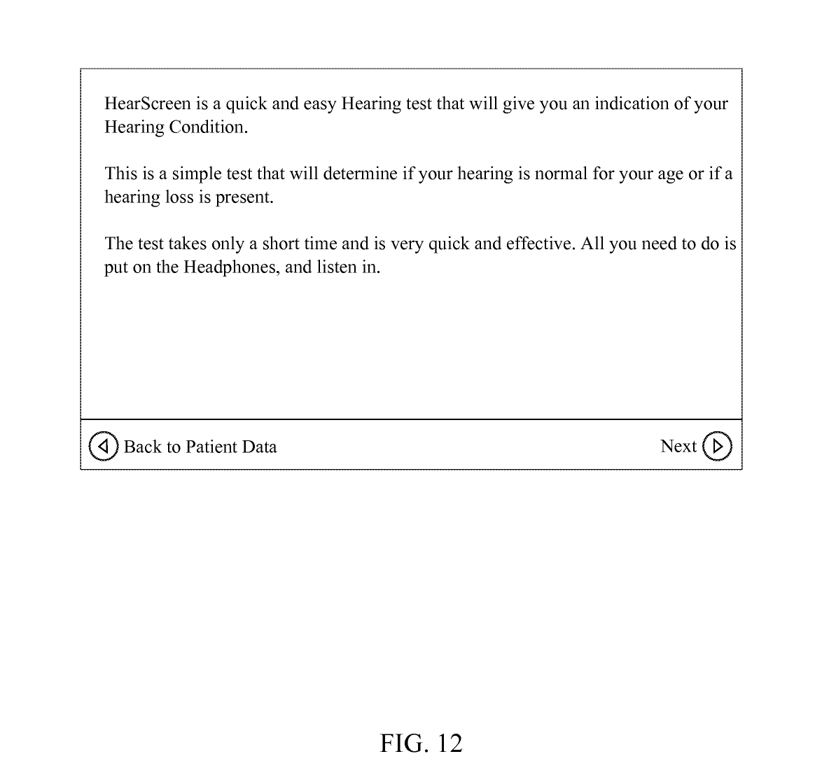

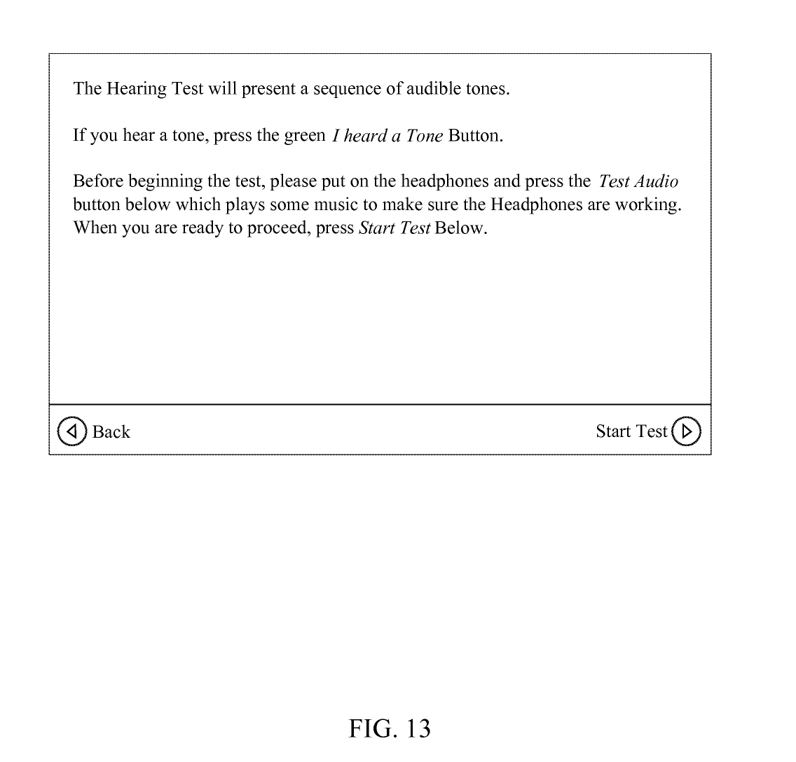

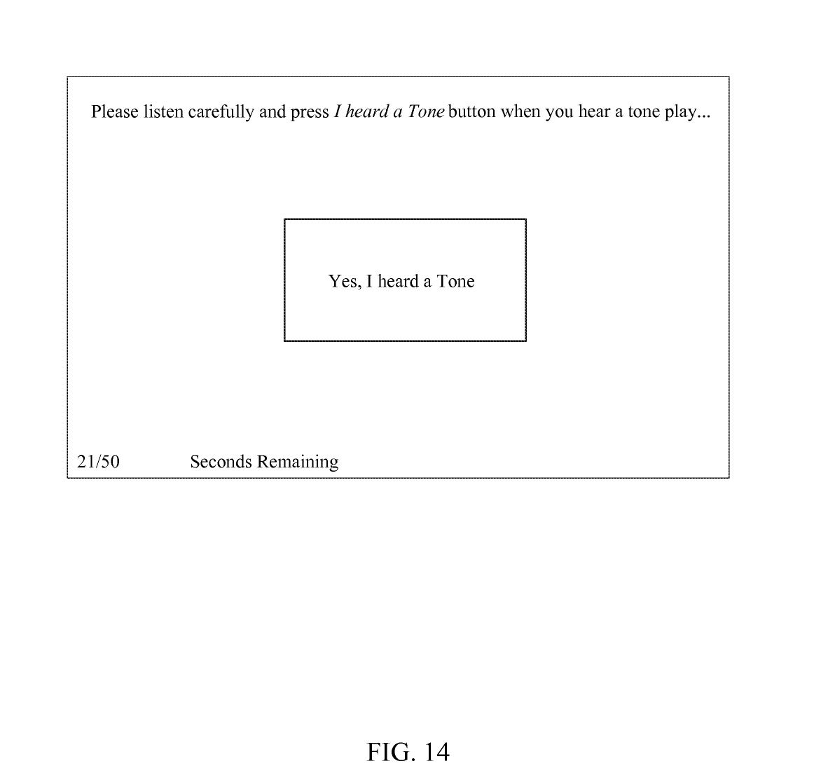

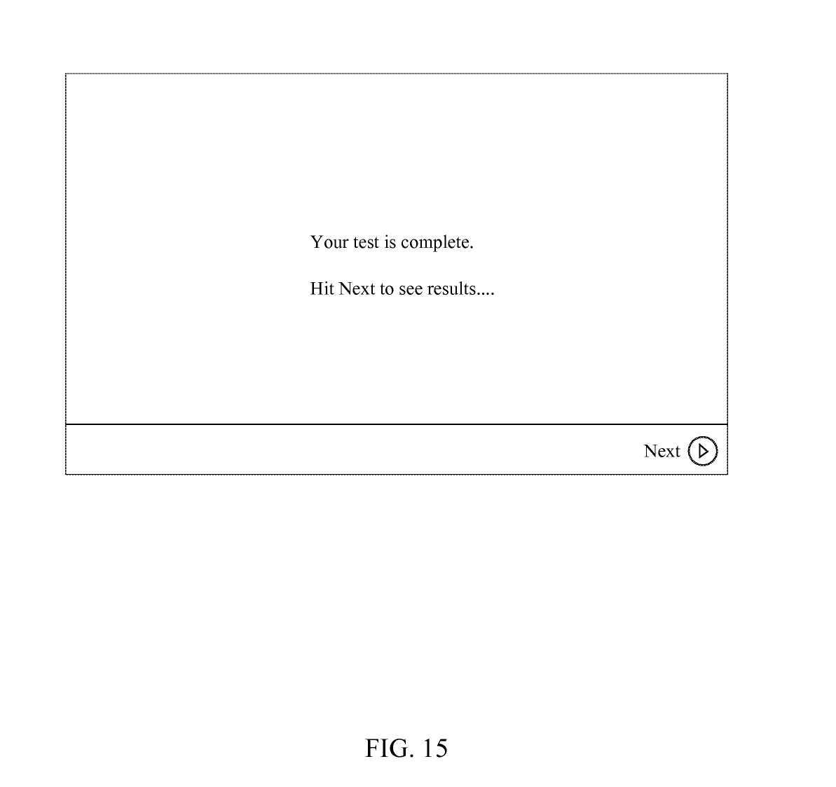

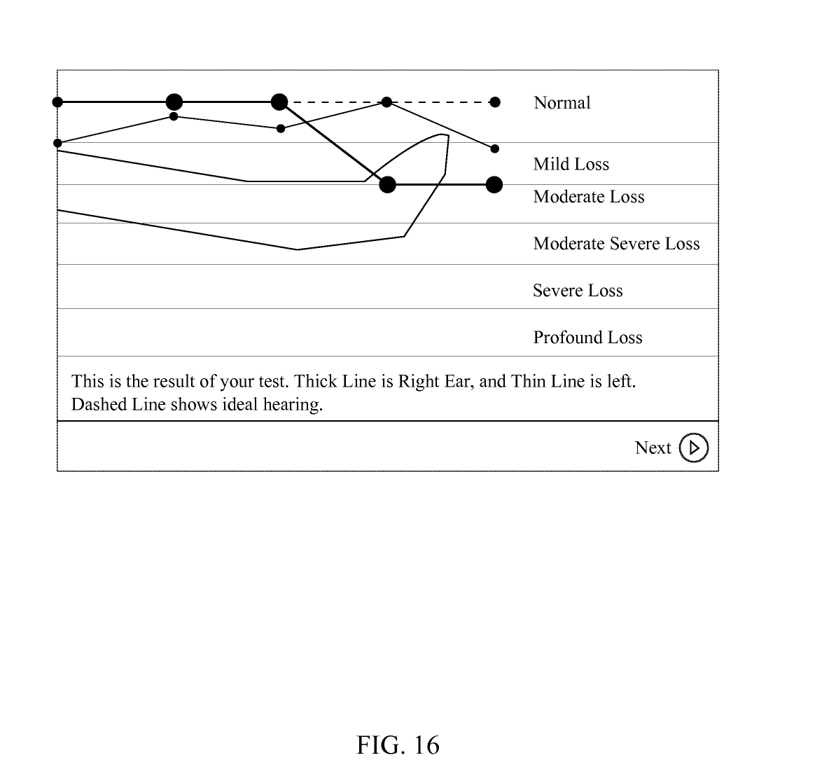

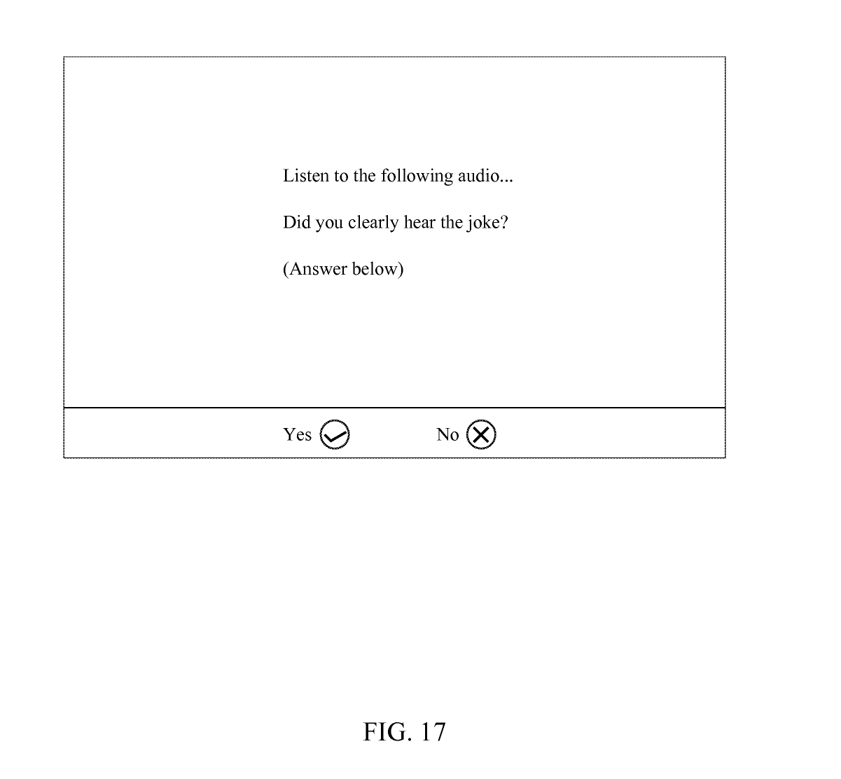

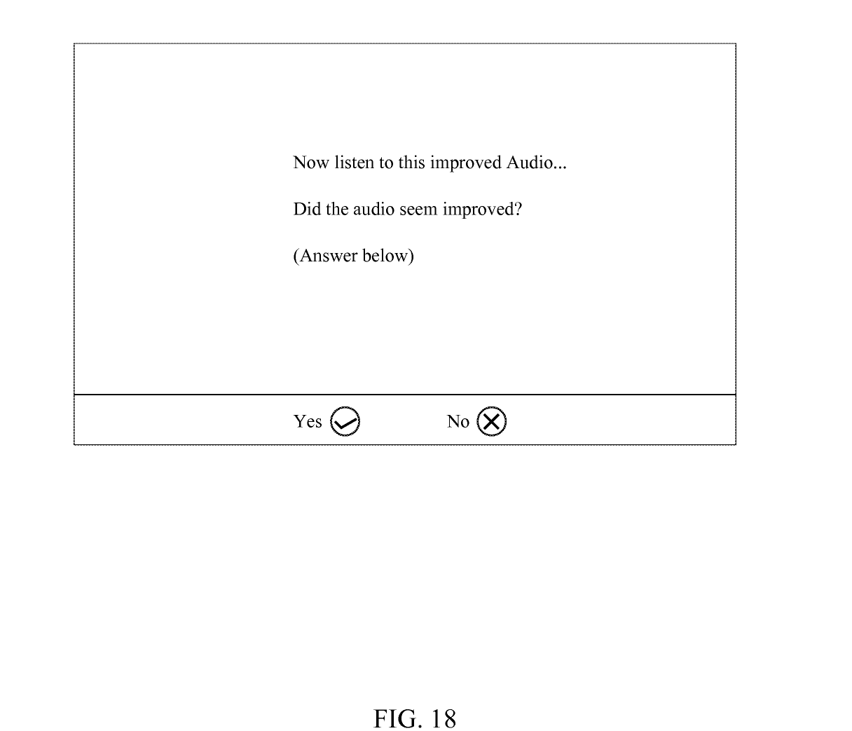

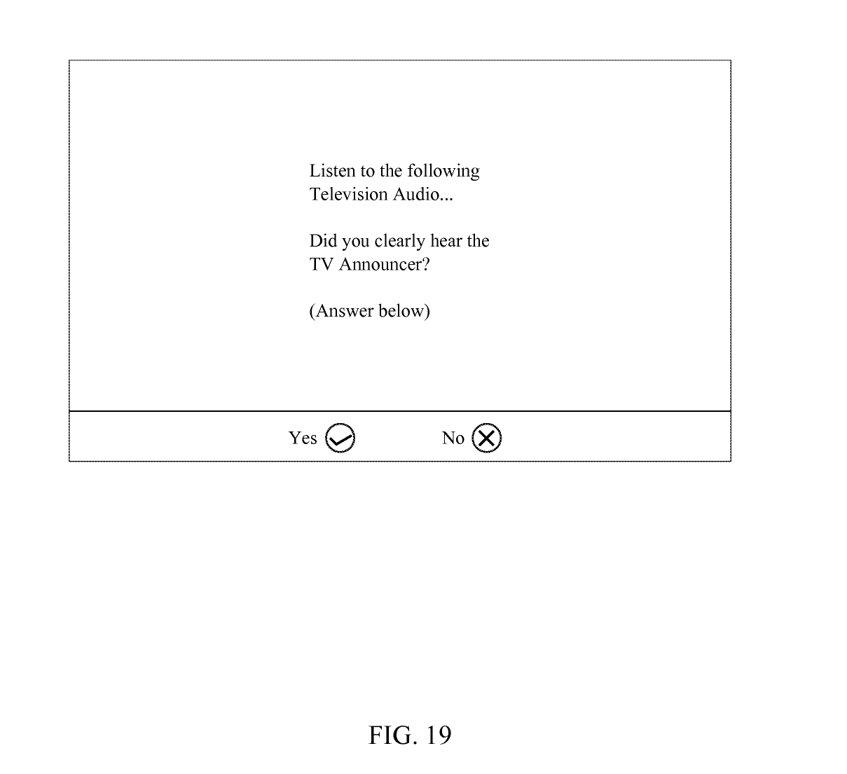

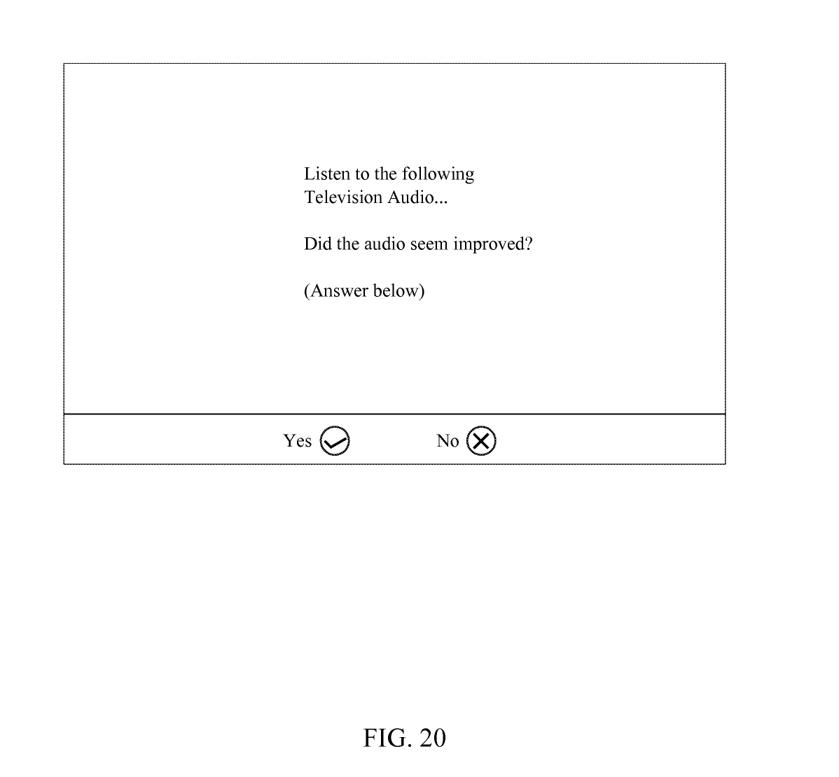

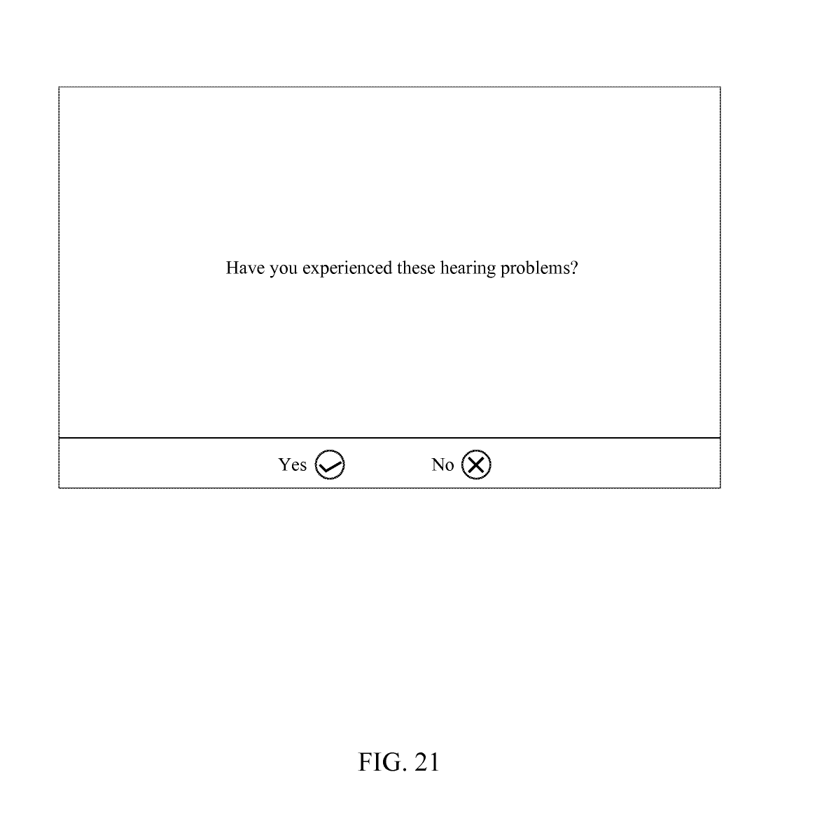

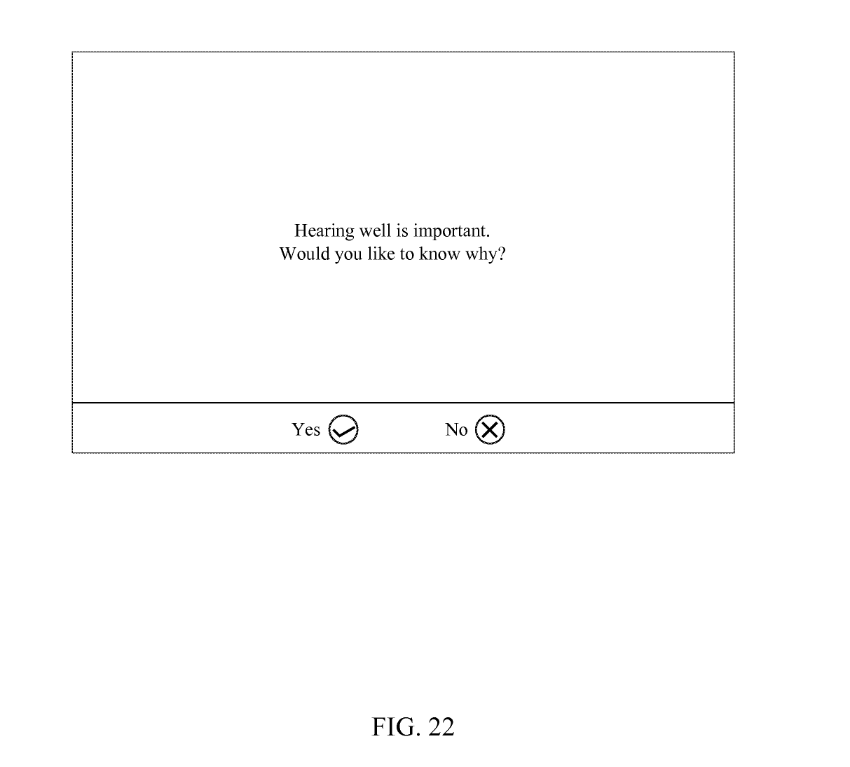

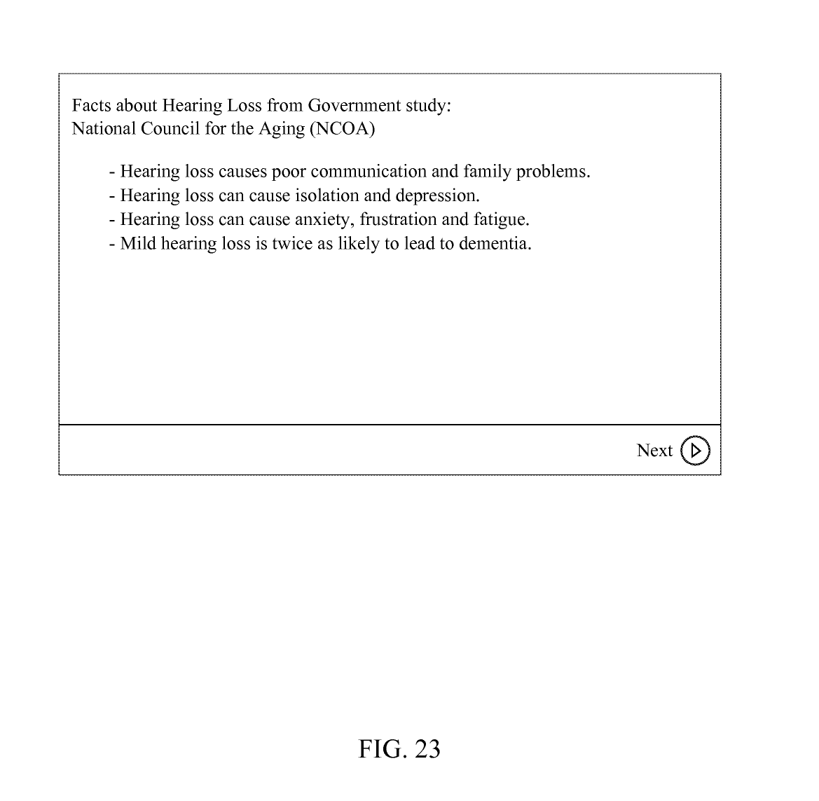

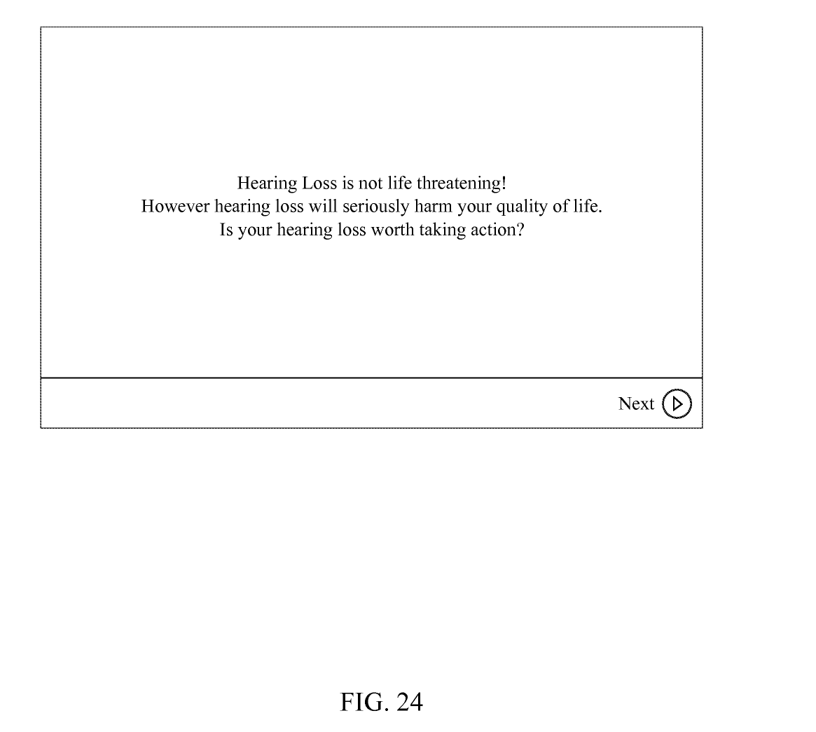

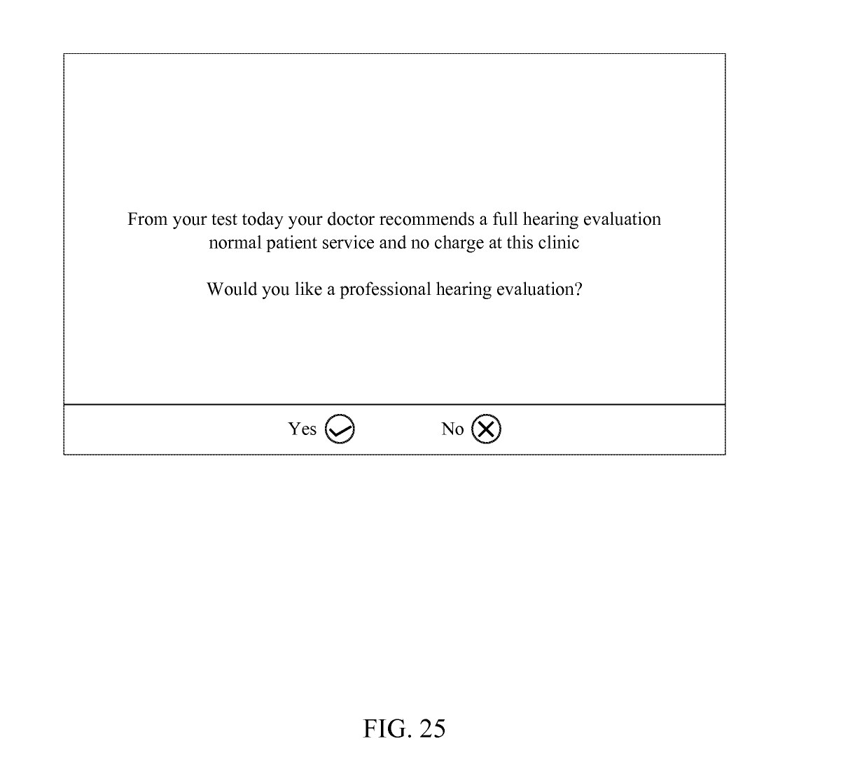

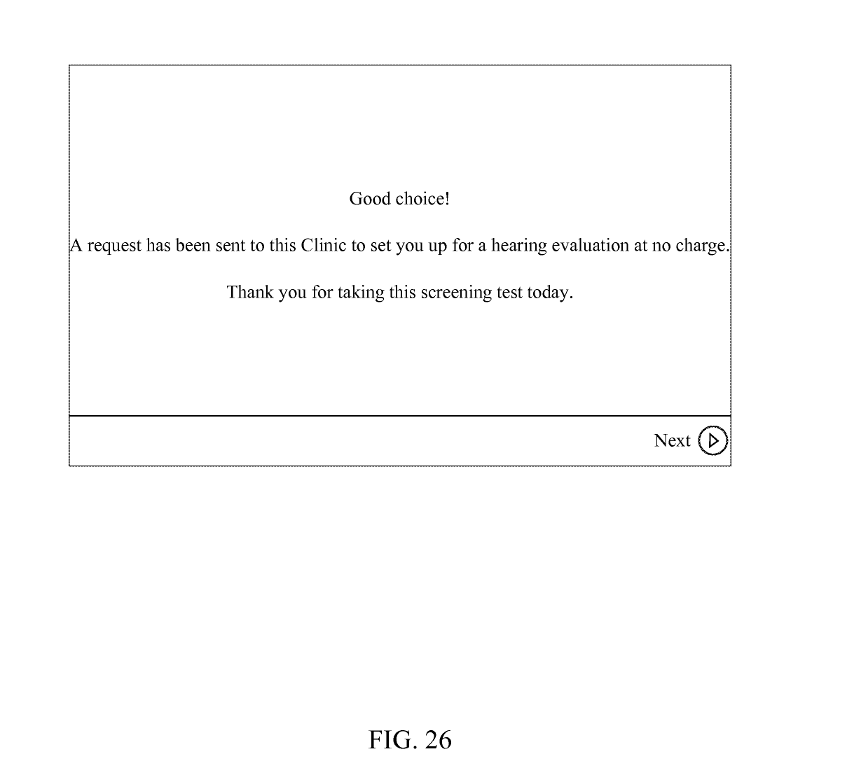

For the second phase, as we know the patients hearing impairment in some detail from the earlier measurement of the patient's hearing loss we construct a “virtual hearing aid algorithm”, so when the patient voluntarily selects the next step, the patient will hear the same original acoustic and visual scenario, with the sound adjusted by the algorithm designed to correct his hearing loss. The recordings will deliver binaural encoded sounds to the individual. The use of this technique leads to a more realistic experience by the listener as both the target speech and ambient noise appear spatially correct, and in particular appear to originate outside of the head. A flowchart of the entire use of the present invention in the preferred implementation is shown in FIGS. 6-8. Example screens of the preferred implementation are shown in FIGS. 9-26.

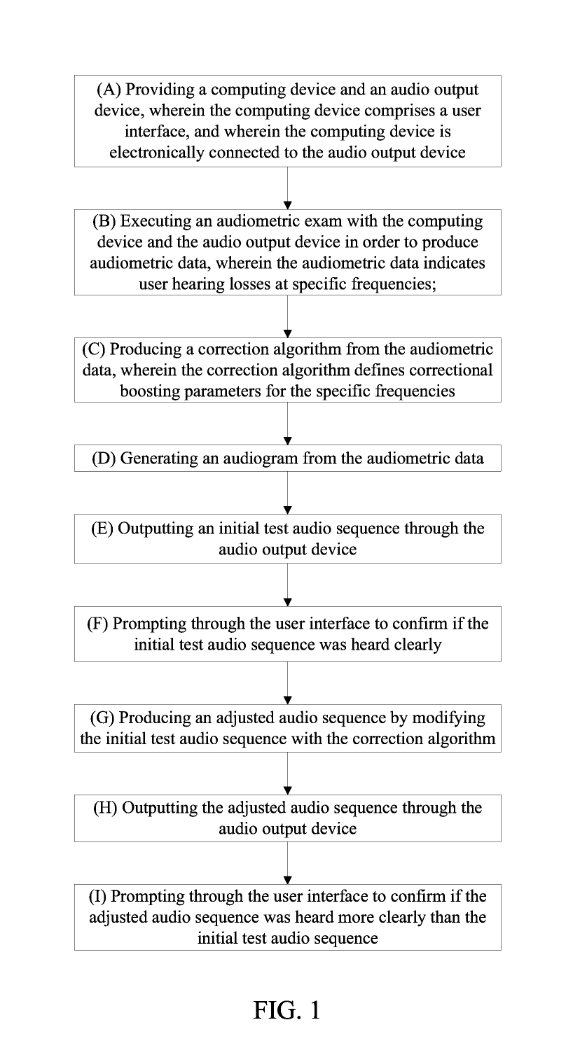

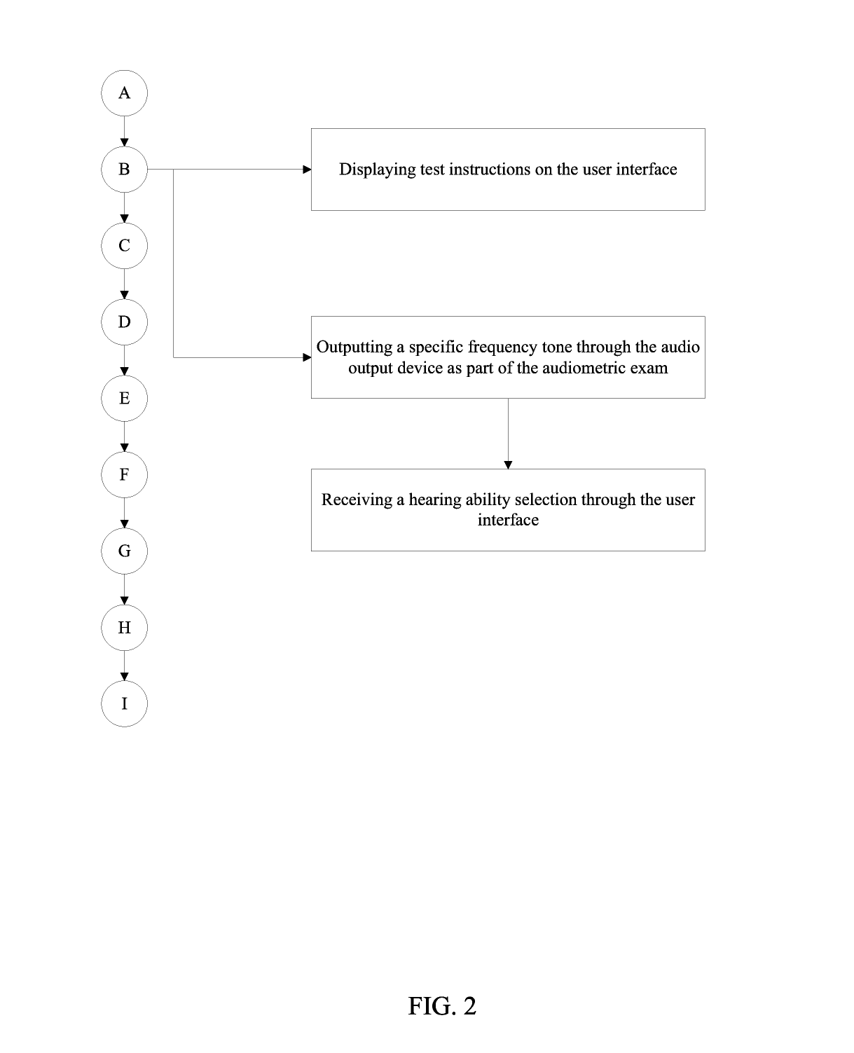

In the general method of the present invention shown in FIG. 1, an audiometric exam is executed with the computing device and the audio output device in order to produce audiometric data. The audiometric data indicates user hearing losses at specific frequencies. Before the audiometric exam, text instructions are displayed on the user interface to inform the user of how to proceed. In order to administer the audiometric exam, a specific frequency tone is outputted through the audio output device as described in FIG. 2. The user is prompted to indicate whether they were able to hear the specific frequency tone, and a hearing ability selection is received through the user interface to indicate as such. This process is repeated for multiple frequencies in order to adequately ascertain the amount of hearing impairment in the user across the typical human hearing frequency ranges. General patient data such as, but not limited to, patient name, age, gender or other relevant attributes may be collected before or after the audiometric exam, or at any other time as desired.

The computing device of the present invention may be given to the patient at the outset of his visit to a physician by the front desk staff in the patient waiting room along with some brief instructions. The patients name and other, maybe coded, details are entered before giving the instrument to the patient. The total test is completed within about 10 or so minutes. When the test is completed two results are known; firstly the audiogram type data which is necessary for the patients base line EMR records and secondly the ‘denial” result and patients voluntary election, or not, to undergo a full acoustic evaluation.

A correction algorithm is then produced from the audiometric data. The correction algorithm defines correctional boosting parameters for the specific frequencies at which the user has hearing loss. The correctional algorithm is a well-known technique for producing hearing aids, and may be derives from any one of a number of prescriptive formulas such as, but not limited to, the National Acoustic Labs (NAL) formula, the Desired Sensation Level input/output (DSLio) formula, or another relevant formula.

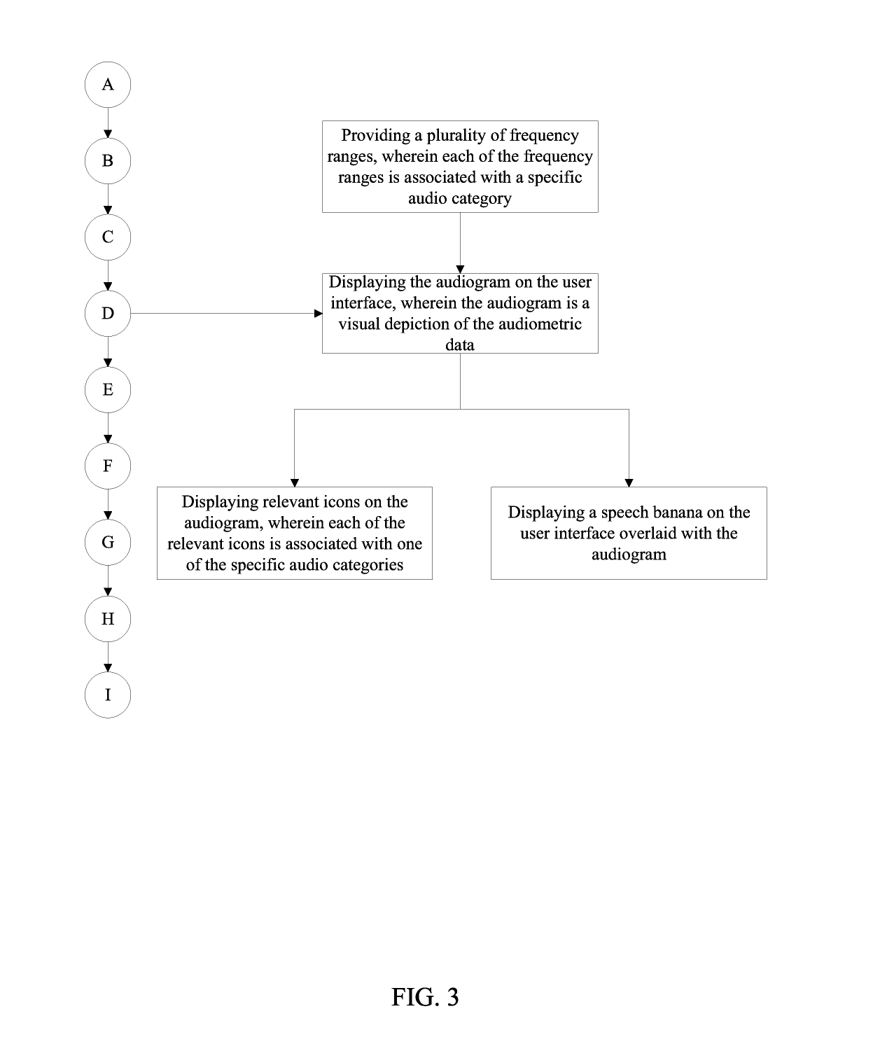

Referring to FIG. 3, an audiogram is also produced from the audiometric data. The audiogram is also a well-known technique for visually depicting hearing loss as ascertained with the audiometric data. The audiogram is displayed on the user interface after the audiometric exam is complete to inform the user visually and accurately of their current state of hearing loss. In the preferred embodiment, a plurality of frequency ranges are defined, wherein each of the frequency ranges is associated with a specific audio category. The audio categories should relate to typical sounds a person might encounter on a day-to-day basis, such as, but not limited to, leaves rustling, birds chirping, human speech, cars passing on the street, and other such typical sounds. Relevant icons are displayed on the audiogram, wherein each of the relevant icons is associated with one of the specific audio categories. In a non-limiting example, a leaf icon is associated with a frequency range associated with rustling leaves, and the leaf icon is displayed on the audiogram in an appropriate location on the frequency axis of the audiogram. This gives the user an additional indication of how their life may be impacted by hearing loss, if the audiometric data indicates that the user has hearing loss in one of the said frequency ranges. Additionally or alternatively, in the preferred embodiment, a “speech banana” is displayed on the user interface overlaid with the audiogram. The speech banana is an audiology industry term referring to the frequency and/or volume domain most common for human speech understanding.

In the preferred embodiment of the present invention, the user is presented with a simplified audiogram, designed to graphically explain the patient's loss scenario in a manner easily understood by the layman. The graph presented is similar to a conventional audiogram in that it has frequency on the horizontal axis and loss charted on the vertical axis. In order to make the graph easily and quickly understood we present overlaid graphics on the graph such as the speech banana as well as relevant icons positioned on the graph indicative of their frequency and volume characteristics. While these concepts have been used in audiology before, we bring novelty to the idea by making this an automated multimedia presentation specifically designed to enlighten the patient based on their test results and help them fully understand their hearing quality.

After the audiogram is displayed, in the preferred embodiment the user is presented with one or more scenarios which attempt illustrate the real-life consequences of hearing loss. The audiometric data which was recorded from the first phase of the test is used to construct acoustic scenarios which the hearing impaired person may find difficult to hear and understand, such as in a real life situation. Examples may be a group discussion or a news flash or similar, all with normal ambient noise, which may remind the patient of the confusion he is experiencing due to the characteristics of his hearing impairment. This test is realistic and more importantly private, because the patient actually confronts his “denial” in private.

To this end, an initial test audio sequence is output through the audio output device. The initial test audio sequence should be generally and easily relatable to; therefore, preferred embodiments of the initial test audio sequence are a person telling a joke in a social setting, a newscaster breaking a story, leaves rustling, birds chirping, a child laughing, or another scenario. However, the present invention should not be limited to the aforementioned scenarios. After the initial test audio sequence is played, the user is prompted through the user interface to confirm if the initial test audio sequence was heard clearly.

An adjusted audio sequence is produced by modifying the initial test audio sequence with the correction algorithm. Similar to the initial test audio sequence, the adjusted audio sequence is outputted through the audio output device, and the user is prompted through the user interface to confirm if the adjusted audio sequence was heard more clearly than the test audio sequence. The user's responses to the initial test audio sequence and the adjusted audio sequence should be recorded and stored in a database for record keeping. In some embodiments, these responses may be utilized to modify aspects of the present invention, such as prompting the user to answer further questions, or displaying certain information.

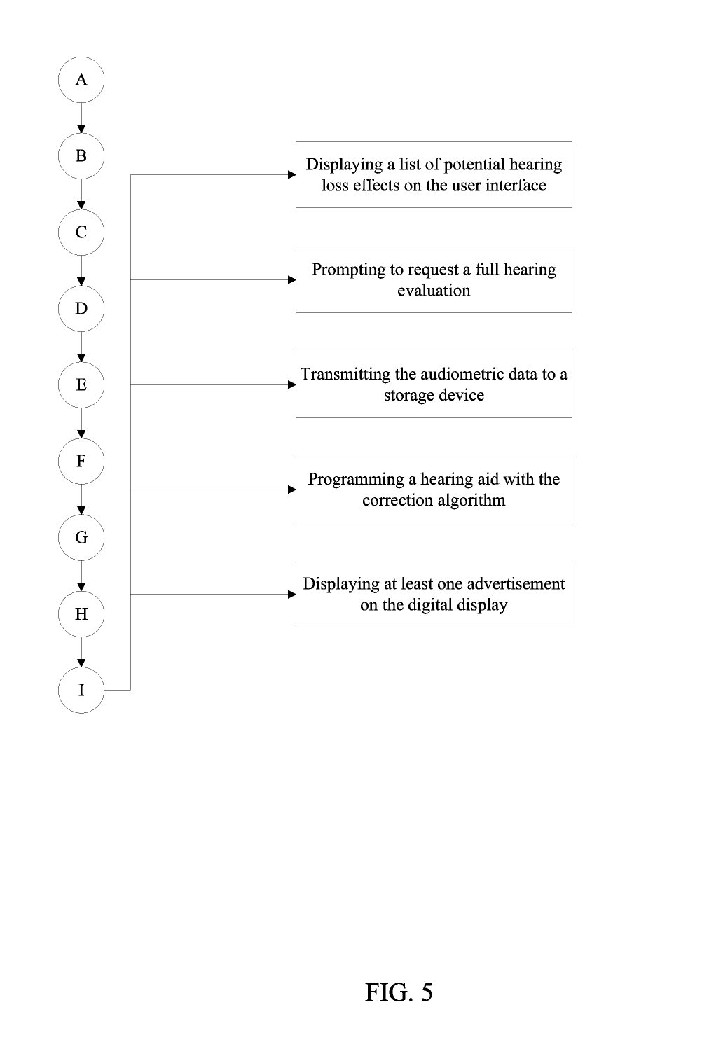

Referring to FIG. 5, after the test audio sequence demonstration, in the preferred embodiment of the present invention the user is presented with additional information about hearing loss. More specifically, a list of potential hearing loss effects is displayed on the user interface. Items on the list of potential hearing loss effects may include, but are not limited to: poor communication and family problems, isolation and depression, anxiety, frustration and fatigue, and possible dementia. The patient may also choose to be shown other well researched government level data from the National Council for the Aging (NCOA) on the long term effects of untreated hearing loss. This yes no decision procedure ultimately guides the patient to voluntarily request a full hearing evaluation. Most importantly this procedure is followed by the person under test is completely in private with no external influence and it is the persons (patient's) decision to select each next step.

Subsequently, the user is prompted to request a full hearing evaluation. If the user chooses to request a full hearing evaluation, the request is sent over a network connection to a relevant destination. The relevant destination is a separate computing device associated with an entity capable of administering or scheduling a full hearing evaluation, such as, but not limited to, a hearing clinic. More specifically, in the preferred embodiment the computing device is designed to send the audiogram, by protected WIFI or other means of transmittal, to any station within, or outside the medical practice, carefully following the patient privacy rules enforced by HIPAA within the EMR (electronic medical record) system operated by the medical practice. Additionally, the present invention may also be used in any location such as an office, a mall, in a store of such as a Walgreens pharmacy, or elsewhere.

The audiometric data collected by the present invention may be useful for purposes outside a simple screening. The audiometric data and/or correction algorithm may be transmitted to a storage device for record keeping purposes or for the purposes of programming a hearing aid with the correction algorithm. This may prove useful for the purpose of a quick, easy and cheap means of remedying hearing loss. A user may walk into a clinic or another type of store of location equipped with the present invention, go through the process of the present invention, and receive a customized over the counter hearing aid in a matter of minutes. At some point during the screening process, the present invention may be used to display at least one advertisement on the digital display. The advertisement(s) may be relevant to prompting the user to purchase a hearing aid, or the advertisement(s) may be completely irrelevant to the present invention.

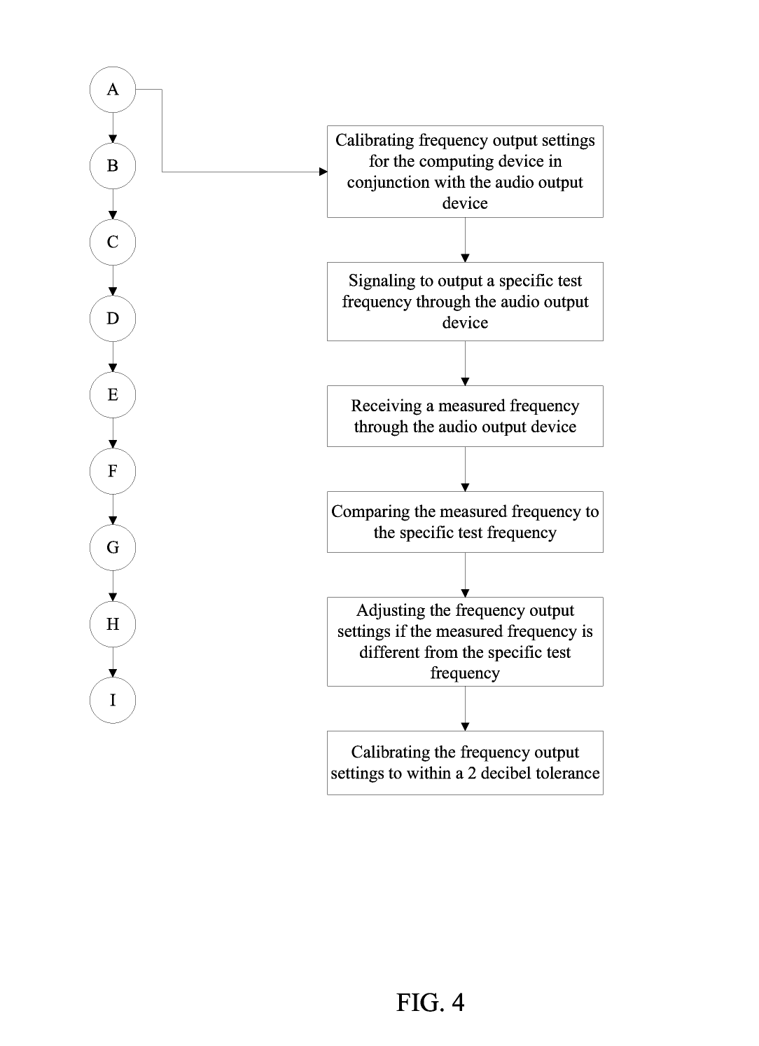

Referring to FIG. 4, in the preferred embodiment of the present invention frequency output settings are calibrated for the computing device in conjunction with the audio output device. To achieve the desired accuracy the ambient sound levels in the ear canal during testing must be low enough so as to have minimal interaction with the test signal. Thus either sealed earphones or noise cancelling technology should be used. As an additional quality control measure the microphone contained in the tablet can be used with appropriate software and factory calibration to measure the ambient noise levels during testing. This data can then be recorded with the patient data, or even used more immediately to alert the user of reduced accuracy.

Acoustic Calibration of the tablet headphone combination can be performed according to an appropriate Audiometer test standard, such as ANSI S3.6 “Specification for Audiometers”.

Additional acoustic calibration such as Headphone equalization can be performed on an acoustic manikin utilizing ear simulators, which approximate the acoustic load of the human external ear. KEMAR is one such manikin. Headphone equalization can be used to improve the accuracy of the binaural playback signals.

Specifically, the frequency output setting should be calibrated to within a 2 decibel tolerance range of desired output versus real output through the audio output device. To perform the calibration, a specific test frequency is signaled to output through the audio output device from the computing device. A measured frequency is then received by an audio measuring device through the audio output device, and the measured frequency is compared to the specific test frequency. The frequency output settings are then adjusted if the measured frequency is different enough from the specific test frequency; specifically, by 2 decibels.

Although the invention has been explained in relation to its preferred embodiment, it is to be understood that many other possible modifications and variations can be made without departing from the spirit and scope of the invention as hereinafter claimed.

What is claimed is:

1. A method of method of acoustic screening for processing hearing loss patients by executing computer-executable instructions stored on a non-transitory computer-readable medium by executing computer-executable instructions stored on a non-transitory computer-readable medium comprises the steps of:
providing a computing device and an audio output device, wherein the computing device comprises a user interface, and wherein the computing device is electronically connected to the audio output device;
executing an audiometric exam with the computing device and the audio output device in order to produce audiometric data, wherein the audiometric data indicates user hearing losses at specific frequencies;
producing a correction algorithm from the audiometric data, wherein the correction algorithm defines correctional boosting parameters for the specific frequencies;
generating an audiogram from the audiometric data;
outputting an initial test audio sequence through the audio output device;
prompting through the user interface to confirm if the initial test audio sequence was heard clearly;
producing an adjusted audio sequence by modifying the initial test audio sequence with the correction algorithm;
outputting the adjusted audio sequence through the audio output device; and
prompting through the user interface to confirm if the adjusted audio sequence was heard more clearly than the initial test audio sequence.

2. A method of method of acoustic screening for processing hearing loss patients by executing computer-executable instructions stored on a non-transitory computer-readable medium by executing computer-executable instructions stored on a non-transitory computer-readable medium as claimed in claim 1 comprises the step of:
displaying test instructions on the user interface.

3. A method of method of acoustic screening for processing hearing loss patients by executing computer-executable instructions stored on a non-transitory computer-readable medium by executing computer-executable instructions stored on a non-transitory computer-readable medium as claimed in claim 1 comprises the steps of:
outputting a specific frequency tone through the audio output device as part of the audiometric exam; and
receiving a hearing ability selection through the user interface, wherein the hearing ability selection indicates whether a user is able to hear the specific frequency tone.

4. A method of method of acoustic screening for processing hearing loss patients by executing computer-executable instructions stored on a non-transitory computer-readable medium by executing computer-executable instructions stored on a non-transitory computer-readable medium as claimed in claim 1 comprises the step of:
displaying the audiogram on the user interface, wherein the audiogram is a visual depiction of the audiometric data.

5. A method of method of acoustic screening for processing hearing loss patients by executing computer-executable instructions stored on a non-transitory computer-readable medium by executing computer-executable instructions stored on a non-transitory computer-readable medium as claimed in claim 5 comprises the steps of:
providing a plurality of frequency ranges, wherein each of the frequency ranges is associated with a specific audio category; and
displaying relevant icons on the audiogram, wherein each of the relevant icons is associated with one of the specific audio categories.

6. A method of method of acoustic screening for processing hearing loss patients by executing computer-executable instructions stored on a non-transitory computer-readable medium by executing computer-executable instructions stored on a non-transitory computer-readable medium as claimed in claim 5 comprises the step of:
displaying a speech banana on the user interface overlaid with the audiogram.

7. A method of method of acoustic screening for processing hearing loss patients by executing computer-executable instructions stored on a non-transitory computer-readable medium by executing computer-executable instructions stored on a non-transitory computer-readable medium as claimed in claim 1 comprises the step of:
calibrating frequency output settings for the computing device in conjunction with the audio output device.

8. A method of method of acoustic screening for processing hearing loss patients by executing computer-executable instructions stored on a non-transitory computer-readable medium by executing computer-executable instructions stored on a non-transitory computer-readable medium as claimed in claim 7 comprises the steps of:
signaling to output a specific test frequency through the audio output device;
receiving a measured frequency through the audio output device;
comparing the measured frequency to the specific test frequency; and
adjusting the frequency output settings, if the measured frequency is different from the specific test frequency.

9. A method of method of acoustic screening for processing hearing loss patients by executing computer-executable instructions stored on a non-transitory computer-readable medium by executing computer-executable instructions stored on a non-transitory computer-readable medium as claimed in claim 7 comprises the step of:
calibrating the frequency output settings to within a 2 decibel tolerance.

10. A method of method of acoustic screening for processing hearing loss patients by executing computer-executable instructions stored on a non-transitory computer-readable medium by executing computer-executable instructions stored on a non-transitory computer-readable medium as claimed in claim 1 comprises the step of:
displaying a list of potential hearing loss effects on the user interface.

11. A method of method of acoustic screening for processing hearing loss patients by executing computer-executable instructions stored on a non-transitory computer-readable medium by executing computer-executable instructions stored on a non-transitory computer-readable medium as claimed in claim 1 comprises the step of:
prompting to request a full hearing evaluation.

12. A method of method of acoustic screening for processing hearing loss patients by executing computer-executable instructions stored on a non-transitory computer-readable medium by executing computer-executable instructions stored on a non-transitory computer-readable medium as claimed in claim 1 comprises the step of:
transmitting the audiometric data to a storage device.

13. A method of method of acoustic screening for processing hearing loss patients by executing computer-executable instructions stored on a non-transitory computer-readable medium by executing computer-executable instructions stored on a non-transitory computer-readable medium as claimed in claim 1 comprises the step of:
programming a hearing aid with the correction algorithm.

14. A method of method of acoustic screening for processing hearing loss patients by executing computer-executable instructions stored on a non-transitory computer-readable medium by executing computer-executable instructions stored on a non-transitory computer-readable medium as claimed in claim 1 comprises the step of:
displaying at least one advertisement on the digital display.
